(19) United States
(12) Patent Application Publication
(10) Pub. No.: US 2010/0053982 A1
(43) Pub. Date: Mar. 4, 2010

(54) Handle

(75) Inventors: Peter Klaus, Emmingen-Liptingen (DE); Martin Winkler, Stockach (DE)

Correspondence Address: THE NATH LAW GROUP, 112 South West Street, Alexandria, VA, 22314, US

(73) Assignee: Berchtold Holding GmbH, Tuttlingen (DE)

(21) Appl. No.: 12/461,825

(22) Filed: Aug. 25, 2009

(30) Foreign Application Priority Data
Aug. 26, 2008 (DE) 10 2008 039 772.5

(51) Int. Cl. F21V 21/06; H05B 41/36; A61B 1/06

(52) U.S. Cl. 362/386; 315/291; 600/249

(57) ABSTRACT

A handle has a grip part rotatably journaled between two end positions and is provided with a sensor which detects one of the end positions.

The present invention relates to a handle for a surgical light in which an illuminated field of the surgical light can be changed by rotating the handle, with the handle including a grip part rotatably journaled between two end positions.

Handles of the kind described above are known from the prior art and serve to vary the size of the illuminated field of the surgical light. With known surgical lights, the rotation of the handle is implemented mechanically so that a light source present inside the surgical light is axially displaced.

It is the object of the present invention to provide a handle of the initially named kind which can be manufactured cost-effectively and with which a simplified adjustment of the illuminated field can be carried out.

This object is satisfied by the features of claim 1 and in particular in that the handle is provided with at least one electrical sensor which detects when a handle has reached one of the two end positions.

It is thus only determined with the handle in accordance with the invention whether one of the end positions is reached, i.e. the total rotary movement of the handle for the adjustment of the illuminated field is not detected. This has the advantage that on a manual repositioning of the surgical light, the illuminated field is not accidentally changed if this is not actually wanted. Only when the handle has been moved into one of the end positions does the electrical sensor output a signal so that an illuminated field adjustment can be initiated.

Advantageous embodiments of the invention are described in the description, in the drawing and in the dependent claims.

In accordance with a first advantageous embodiment, the sensor can be a force sensor. The further possibility hereby results of varying the adjustment of the illuminated field in dependence on the time and/or on the duration of the applied force in order thereby to realize a fast action, for example.

In accordance with a further advantageous embodiment, two sensors can be used, with the handle being pretensioned into a position of rest between the two end positions by a spring means. It can be detected in this manner when the handle is located in the one or the other end position if it has been rotated.

In accordance with a further advantageous embodiment, the sensor can be connected to an electric control with which the illuminated field of the surgical light can be changed, for example in that the brightness of individual light sources of the surgical light are changed. Such an embodiment can in particular be advantageous with surgical lights having LEDs as light sources.

In accordance with a further advantageous embodiment, the sensor can be made such that it delivers a sensor signal which can be changed in dependence on the force applied. In this case, the control can vary the speed at which the illuminated field is changed in dependence on the amplitude of the sensor signal. It is understood that the sensor signal does not necessarily have to be an analog signal, but can also be a digital signal.

In accordance with a further advantageous embodiment, the control cannot trigger any change of the illuminated field when a minimal sensor signal is fallen below and/or when a maximum sensor signal is exceeded. In this embodiment, it is particularly easily ensured that no accidental adjustment of the illuminated field takes place since the sensor signal has to lie within a working range which is defined by the minimal and the maximum sensor signal. It can be advantageous in this respect to determine the dynamics of the movement of the handle so that the control does not, for example, trigger any change of the illuminated field if a predetermined amplitude range of the sensor signal was passed through within a predetermined minimum time. It can namely be assumed in this case that the surgical light is being repositioned and no adjustment of the illuminated field is necessary.

In accordance with a further advantageous embodiment, the control can fix a plurality of sequential amplitude ranges for the sensor signal, with a light field change being triggered at a different speed in each amplitude range, which increases the operating comfort.

In accordance with a further advantageous embodiment, the handle can be connected to a pivot lever which acts on the sensor via a damping element. On the one hand, this contributes to an increased service life and, on the other hand, in turn to an improved operating comfort.

The present invention will be described in the following purely by way of example with reference to an advantageous embodiment and to the enclosed drawings. There are shown:

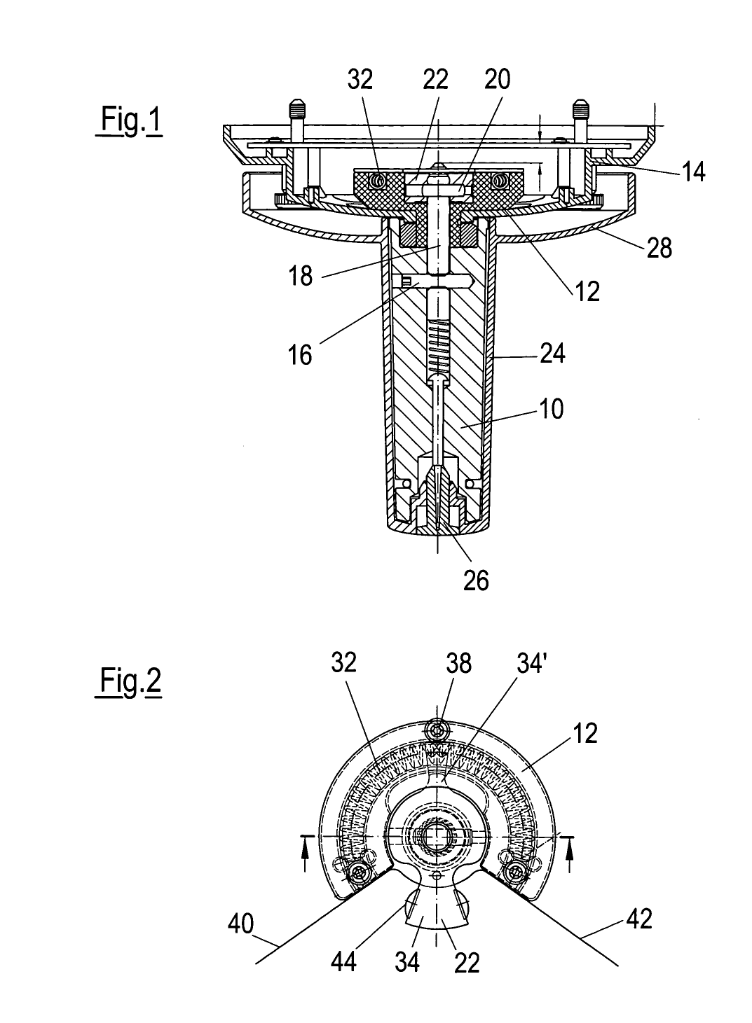

FIG. 1 a longitudinal section through a handle;

FIG. 2 an enlarged plan view of a part of the handle of FIG. 1;

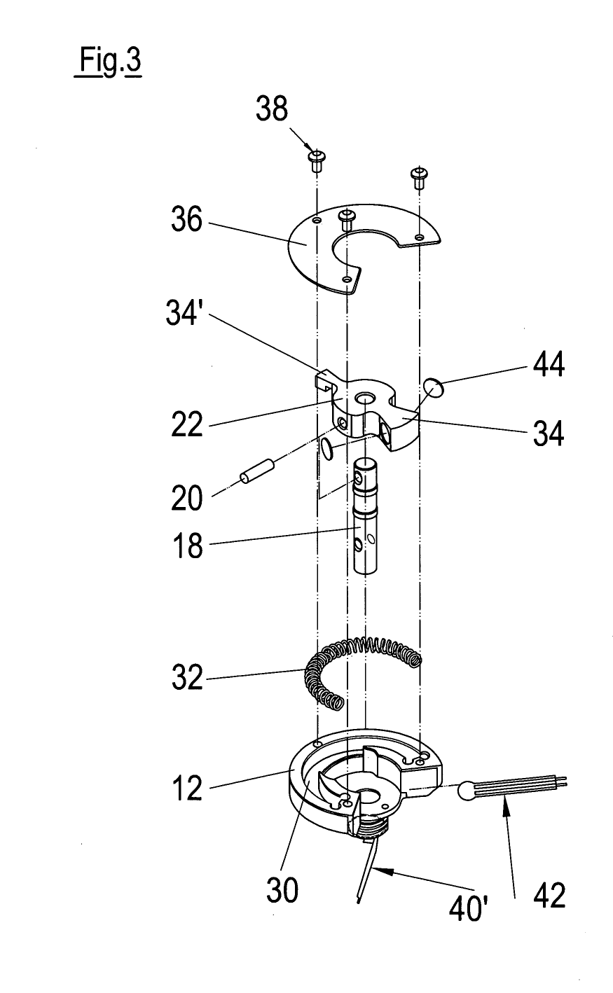

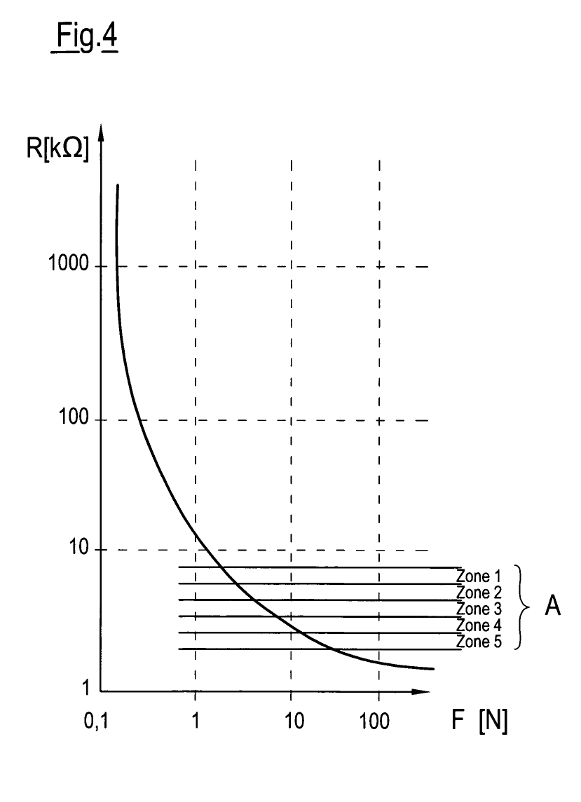

FIG. 3 an exploded drawing of the arrangement of FIG. 2; and

FIG. 4 a characteristic line of the electric sensor.

FIG. 1 shows a sectional view of a handle for a surgical light (not shown) with which the illuminated field can be changed by rotating the handle, for example in that the focus of a light source is changed mechanically or in that the brightness of individual light sources is changed.

The handle includes a grip part 10 which is rotatably journaled between two end positions and which is pivotable by approximately 70° about its longitudinal axis, with the grip part 10 being connected to a holder 12 which can be attached to the surgical light via a fastening shell 14. As the Figure shows, the grip part 10 is connected via a cotter pin 16 to a coupling shaft 18 which is guided through the holder 12 and is rotationally fixedly connected via a cotter pin 20 to a pivot lever 22 which is rotatably received within the holder 12.

The reference numeral 24 in FIG. 1 designates a sterilizable grip sleeve which is pushed onto the grip part 10 and is held at the grip part 10 with the help of a pin 26. In this respect, a dome-like end part 28 which almost completely covers the fastening shell 14 is shaped at the grip sleeve 24.

FIG. 3 shows an exploded view of the assembly of the holder 12 and the pivot lever 22. As can be recognized, a ring groove 30, which extends over approximately 220° and into which a spiral spring 32 is inserted, is located in the holder 12. The pivot lever 22 rotatably journaled within the holder 12 has a front shift pad 34 as well as a diametrically opposed rear prolongation 34′ which is hooked to the spring 32 in the assembled state (cf. FIG. 2). The pivot lever 22 can hereby be rotated around its center axis and against the force of the spring 33 within the holder 12 and the pivot lever 22 always returns to its center position shown in FIG. 2 without any external force effect. A cover 36 which can be connected to the holder 12 via three screws 38 is provided for the fixing of the spring 32 within the holder 12.

As FIGS. 2 and 3 furthermore show, two electrical force sensors 40 and 42 are provided at the holder 12 which are each adhesively bonded to an end-face surface of the holder 12 and which detect an abutment of the shift pad 34. In the region of the abutment surface, two lens-shaped damping elements 44 are provided at the shift pad 34.

As can be seen from the above description, the grip part 10 can be rotated from its center position anticlockwise or clockwise about its center axis, with the pivot lever 22 likewise being rotated on such a rotation. When the pivot lever 22 has reached one of its end positions, it abuts one of the sensors 40 and 42 and exerts a force onto the respective sensor so that the sensor outputs a signal changing in dependence on the exerted force. In dependence on the output signal, the illuminated field of the surgical light is then changed, for which purpose a control (not shown) is provided which will be described in more detail in the following.

The sensors 40 and 42 are made, as described above, as force sensors so that a respective force acts on the respective sensor and changes its resistance by exertion of a torque onto the grip part 10 and on reaching an end position. The control monitors the resistance value of each sensor several times a second and evaluates it. A working region A formed from a plurality of zones (zone 1 to zone 5) is defined on the characteristic line of the sensors, such as is shown by way of example in FIG. 4, for the evaluation. Each zone corresponds to a resistance range, with the individual zones being of equal size and following one another. A specific action can be stored in the control for each zone.

A specific speed at which the illuminated field is changed is associated with each zone for the adjustment of the illuminated field of the surgical light. In this respect, zone 1 stands for the lowest speed and zone 5 for the highest speed at which the illuminated field is changed.

If the surgical light is repositioned with the help of the handle, the characteristic line is passed through at very high speed due to the relatively high force applied. To prevent any unwanted adjustment of the illuminated field size in this case, a time delay starts in the control at every first occurrence of the resistance value in the working region A. Only after the presettable delay has passed is a light field adjustment triggered if the instantaneous resistance value is still within the working region A.

As was described above, a dynamic illuminated field enlargement or reduction can be achieved by rotating the grip part 10 clockwise or counter-clockwise over a defined triggering angle since the time duration of the illuminated field adjustment is fixed over the duration of the actuation of the end abutment and the speed of the illuminated field adjustment is fixed via the intensity of the actuation. On a repositioning of the surgical light with the help of the handle, any unwanted adjustment of the illuminated field is avoided via the above-described characteristic line control. After an actuation of the illuminated field adjustment, the handle is automatically brought into its centre position by releasing the handle so that a defined starting position is always present.

### Reference Numeral List

10 grip part
12 holder
14 fastening shell
16 cotter pin
18 coupling shaft
20 cotter pin
22 pivot lever
24 grip sleeve
26 pin
28 end part
30 ring groove
32 spring
34 shift pad
34′ prolongation
36 cover
38 screws
40, 42 sensor
44 damping elements
A working region

1. A handle for a surgical light in which an illuminated field of the surgical light can be changed by rotating the handle, comprising a grip part (10) rotatably journaled between two end positions, wherein the handle is provided with at least one electrical sensor (40, 42) which detects when the grip part (10) has reached one of the end positions.

2. A handle in accordance with claim 1, wherein the sensor (40, 42) is a force sensor.

3. A handle in accordance with claim 1, wherein two sensors (40, 42) are provided; and wherein the grip part (10) is pre-tensioned by a spring means (32) into a position of rest between the two end positions.

4. A handle in accordance with claim 1, wherein the sensor (40, 42) is connected to an electrical control with which the illuminated field of the surgical light can be changed.

5. A handle in accordance with claim 4, in which the illuminated field of the surgical light can be changed by dimming individual light sources.

6. A handle in accordance with claim 4, wherein the sensor (40, 42) delivers a sensor signal which can be changed in dependence on the force applied; and wherein the control varies the speed at which the illuminated field is changed in dependence on the amplitude of the sensor signal.

7. A handle in accordance with claim 4, wherein the control does not trigger any change of the illuminated field after at least one of falling below a minimal sensor signal and exceeding a maximum sensor signal.

8. A handle in accordance with claim 4, wherein the control does not trigger any change of the illuminated field when a predetermined amplitude range of the sensor signal was passed through within a predetermined minimum time.

9. A handle in accordance with claim 4, wherein the control fixes a plurality of sequential amplitude ranges of the sensor signal, with a light field change being triggered at a different speed in each amplitude range.

10. A handle in accordance with claim 9, wherein the speed increases with each sequential amplitude region.

11. A handle in accordance with claim 1, wherein a pivot lever (22) which acts on the sensor (40, 42) via a damping element (44) is connected to the grip part (10).

12. A medical light having a handle for a surgical light in which an illuminated field of the surgical light can be changed by rotating the handle, comprising a grip part (10) rotatably journaled between two end positions, wherein the handle is provided with at least one electrical sensor (40, 42) which detects when the grip part (10) has reached one of the end positions.
